(19) United States
(12) Patent Application Publication
(10) Pub. No.: US 2025/0010076 A1
(43) Pub. Date: Jan. 9, 2025

(54) PATIENT FEEDBACK SYSTEM FOR ADJUSTING STIMULATION PARAMETERS

(71) Applicant: Boston Scientific Neuromodulation Corporation, Valencia, CA (US)

(72) Inventors: Lisa Denise Moore, Glendale, CA (US); Benjamin Ray Hoenes, St. Louis Park, MN (US)

(21) Appl. No.: 18/762,090

(22) Filed: Jul. 2, 2024

Related U.S. Application Data
(60) Provisional application No. 63/525,116, filed on Jul. 5, 2023.

(51) Int. Cl. A61N 1/36; A61N 1/05; A61N 1/372; A61N 1/375; G16H 10/20; G16H 10/60; G16H 20/40; G16H 50/70

(52) U.S. Cl. CPC A61N 1/36132; A61N 1/0534; A61N 1/37247; A61N 1/3754; G16H 10/20; G16H 10/60; G16H 20/40; G16H 50/70

(57) ABSTRACT

A system may include a stimulator configured to deliver a DBS therapy according to a first therapy program, and a processing system. The processing system may be configured for use to receive a logged event indicative of an acute reduction in therapy, automatically identify a second therapy program for the DBS therapy to address the acute reduction in therapy, deliver the DBS therapy according to the second therapy program, prompt the user to report whether the user likes or dislikes the DBS therapy delivered according to the second therapy program, and control the DBS therapy based on the report by choosing a third therapy program, reverting to the first therapy program, or continuing to deliver the DBS therapy according to the second therapy program.

### CLAIM OF PRIORITY

This application claims the benefit of U.S. Provisional Application No. 63/525,116 filed on Jul. 5, 2023, which is hereby incorporated by reference in its entirety.

### TECHNICAL FIELD

This document relates generally to medical systems, and more particularly, but not by way of limitation, to systems, devices, and methods for delivering therapy using feedback to address acute reductions in therapy.

### BACKGROUND

Medical devices may include therapy-delivery devices configured to deliver a therapy to a patient and/or monitors configured to monitor a patient condition via user input and/or sensor(s). For example, therapy-delivery devices for ambulatory patients may include wearable devices and implantable devices, and further may include, but are not limited to, stimulators (such as electrical, thermal, or mechanical stimulators) and drug delivery devices (such as an insulin pump). An example of a wearable device includes, but is not limited to, transcutaneous electrical neural stimulators (TENS), such as may be attached to glasses, an article of clothing, or a patch configured to be adhered to skin. Implantable stimulation devices may deliver electrical stimuli to treat various biological disorders, such spinal cord stimulators (SCS) to treat chronic pain, cortical and Deep Brain Stimulators (DBS) to treat motor and psychological disorders, Peripheral Nerve Stimulation (PNS), Functional Electrical Stimulation (FES), and other neural stimulators to treat urinary incontinence, sleep apnea, shoulder subluxation, etc. By way of example and not limitation, a DBS system may be configured to treat tremor, bradykinesia, and dyskinesia and other motor disorders associated with Parkinson's Disease (PD).

Closed loop stimulation adjustment is becoming an expected feature in next generation systems. There are closed loop systems that rely on internal physiological sensing or longer-term external sensing. However, these systems may not be able to counter an acute reduction in therapeutic outcome because the system may not have the full context, from sensing alone, of what is really leading to the reduction in therapeutic outcome. For example, a patient may quickly develop a problem with eating dinner, even though the patient had no trouble cating lunch. Examples of an acute reduction in therapy include a quick reduction in relief in a symptom that is being treated by the therapy, a quick worsening side effect when the therapy is delivered, or a quickly-developed problem with an activity performed by the patient. There is a need in the art for improved therapy system to address such acute reductions in therapy.

### SUMMARY

Embodiments of the present subject matter provide improved systems for counteracting an acute reduction in therapeutic outcome. Some embodiments provide a system capable of using user logging, such as log(s) entered by a patient and/or caregiver, to respond to acute reductions in therapy, identify patterns of therapy reduction, and develop novel event related adjustments for patients. The feedback may include current symptoms, side effects and patient activities to adjust weighting for symptoms/side effects and select a stimulation program. The stimulation program may be a tested and saved program in the stimulator system. DBS is provided as an example of a stimulator system. However, the present subject matter may be implemented with other medical device systems, including other neural stimulation systems such as SCS, PNS, FES, and TENS.

An example (e.g., “Example 1”) of a system may include a deep brain stimulator (DBS) configured to deliver a DBS therapy to a patient according to a first therapy program, and a processing system. The processing system may be configured for use to receive a logged event from a user or sensor indicative of an acute reduction in therapy, automatically identify a second therapy program for the DBS therapy to address the acute reduction in therapy, deliver the DBS therapy according to the second therapy program, prompt the user to report whether the user likes or dislikes the DBS therapy delivered according to the second therapy program, and control the DBS therapy based on the report. Controlling the DBS therapy based on the report may include choosing a third therapy program when the user does not like the second therapy program, reverting to the first therapy program when the user does not like the second therapy program, or continuing to deliver the DBS therapy according to the second therapy program when the user likes the second therapy program.

In Example 2, the subject matter of Example 1 may optionally be configured such that the logged event is indicative of a symptom known to the user that may be experienced by the patient and that is known to potentially cause the acute reduction in therapy, and the automatically identified second therapy program is an accessible program for targeting the symptom.

In Example 3, the subject matter of Example 1 may optionally be configured such that the logged event is indicative of a side effect known to the user that may be experienced by the patient and that is known to the user to potentially cause the acute reduction in therapy, and the automatically identified second therapy program is an accessible program for targeting the side effect.

In Example 4, the subject matter of Example 1 may optionally be configured such that the logged event from the user is indicative of an activity that corresponds to a set of symptoms or side effects that cause the first therapy to be suboptimal.

In Example 5, the subject matter of Example 4 may optionally be configured such that the processing system is further configured for use to automatically request additional information when the logged event is received. The additional information may be requested using user-answered queries or may be received via user-provided free text.

In Example 6, the subject matter of any one or more of Examples 1-5 may optionally be configured such that a weighted summary map combines weights of clinical effects data into a cumulative score, and the second therapy program is automatically identified by altering the weights of symptoms in the clinical effects data, and identifying a program with a best estimated score.

In Example 7, the subject matter of Example 6 may optionally be configured such that the second therapy program is automatically identified by placing a side effect barrier on the cumulative map, and the automatically identified second therapy program has a best estimated score remaining outside of a side effect region.

In Example 8, the subject matter of any one or more of Examples 1-7 may optionally be configured such that the processing system is further configured for altering a workflow to simplify patient interaction when the logged event is repeatedly received.

In Example 9, the subject matter of Example 8 may optionally be configured such that the workflow is altered by introducing a schedule for delivering the DBS according to the second therapy schedule.

In Example 10, the subject matter of any one or more of Examples 1-9 may optionally be configured such that the processing system is further configured for determining if a program limit has been reached the DBS therapy is delivered according to another therapy program, and only delivering the DBS therapy according to the other therapy program when a program limit has not been reached.

In Example 11, the subject matter of any one or more of Examples 1-10 may optionally be configured such that the user includes at least one of the patients or a caregiver for the patient.

In Example 12, the subject matter of any one or more of Examples 1-11 may optionally be configured such that the processing system is further configured for receiving a report on the second therapy, by prompting the user or automatically sensing a detectable response, within a first time period after beginning to deliver the DBS therapy according to the second therapy, and continuing to deliver the DBS therapy according to the second therapy program when the report is good. The processing system may further be configured for determining whether to continue to deliver the DBS therapy according to the second therapy program within a second time period after beginning to deliver the DBS therapy based on another report by prompting the user or automatically sensing the detectable response or another detectable response. The second time period may be longer than the first time period.

In Example 13, the subject matter of Example 12 may optionally be configured such that at least one of the first time period or the second time period depends on an event type for the logged event.

In Example 14, the subject matter of any one or more of Examples 12-13 may optionally be configured such that the processing system is further configured for adding at least one prompt for the user to report on the second therapy within at least one time period longer than the second time period.

In Example 15, the subject matter of any one or more of Examples 1-14 may optionally be configured such that the processing system is further configured for receiving additional logged or sensed events and using the logged or sensed events to identify patterns of therapy reduction and develop event-related adjustments to the DBS therapy for the identified patterns.

Example 16 includes subject matter (such as a method, means for performing acts, machine readable medium including instructions that when performed by a machine cause the machine to perform acts, or an apparatus to perform). The subject matter may include delivering a deep brain stimulation (DBS) therapy to a patient according to a first therapy program, receiving a logged event from a user or sensor indicative of an acute reduction in therapy, automatically identifying a second therapy program for the DBS therapy to address the acute reduction in therapy, delivering the DBS therapy according to the second therapy program, prompting the user to report whether the user likes or dislikes the DBS therapy delivered according to the second therapy program, and controlling the DBS therapy based on the report by choosing a third therapy program when the user does not like the second therapy program, reverting to the first therapy program when the user does not like the second therapy program, or continuing to deliver the DBS therapy according to the second therapy program when the user likes the second therapy program.

In Example 17, the subject matter of Example 16 may optionally be configured such that the logged event is indicative of a symptom known to the user that may be experienced by the patient and that is known to potentially cause the acute reduction in therapy.

In Example 18, the subject matter of Example 17 may optionally be configured such that the automatically identified second therapy program is an accessible program for targeting the symptom.

In Example 19, the subject matter of Example 16 may optionally be configured such that the logged event is indicative of a side effect known to the user that may be experienced by the patient and that is known to the user to potentially cause the acute reduction in therapy.

In Example 20, the subject matter of Example 19 may optionally be configured such that the automatically identified second therapy program is an accessible program for targeting the side effect.

In Example 21, the subject matter of Example 16 may optionally be configured such that the logged event from the user is indicative of an activity that corresponds to a set of symptoms that cause the first therapy to be suboptimal.

In Example 22, the subject matter of Example 21 may optionally be configured to further include automatically requesting additional information when the logged event is received, wherein the additional information is requested using user-answered queries or is received via user-provided free text.

In Example 23, the subject matter of any one or more of Examples 16-22 may optionally be configured such that a weighted summary map combines weights of clinical effects data into a cumulative score, and the second therapy program is automatically identified by altering the weights of symptoms in the clinical effects data, and identifying a program with a best estimated score.

In Example 24, the subject matter of Example 23 may optionally be configured such that the second therapy program is automatically identified by placing a side effect barrier on the cumulative map, the automatically identified second therapy program has a best estimated score remaining outside of a side effect region.

In Example 25, the subject matter of any one or more of Examples 16-24 may optionally be configured to further include altering a workflow to simplify patient interaction when the logged event is repeatedly received.

In Example 26, the subject matter of Example 25 may optionally be configured such that the workflow is altered by introducing a schedule for delivering the DBS according to the second therapy schedule.

In Example 27, the subject matter of any one or more of Examples 16-26 may optionally be configured to further include determining if a program limit has been reached the DBS therapy is delivered according to another therapy program, and only delivering the DBS therapy according to the other therapy program when a program limit has not been reached.

In Example 28, the subject matter of any one or more of Examples 16-27 may optionally be configured such that the user includes at least one of the patients or a caregiver for the patient.

In Example 29, the subject matter of any one or more of Examples 16-28 may optionally be configured to further include receiving a report on the second therapy, by prompting the user or automatically sensing a detectable response, within a first time period after beginning to deliver the DBS therapy according to the second therapy, and continuing to deliver the DBS therapy according to the second therapy program when the report is good. The subject matter may further include determining whether to continue to deliver the DBS therapy according to the second therapy program within a second time period after beginning to deliver the DBS therapy based on another report by prompting the user or automatically sensing the detectable response or another detectable response. The second time period may be longer than the first time period.

In Example 30, the subject matter of Example 29 may optionally be configured such that at least one of the first time period or the second time period depends on an event type for the logged event.

In Example 31, the subject matter of any one or more of Examples 30-31 may further include adding at least one prompt for the user to report on the second therapy within at least one time period longer than the second time period.

In Example 32, the subject matter of any one or more of Examples 16-31 may further include receiving additional logged or sensed events and using the logged or sensed events to identify patterns of therapy reduction and develop event-related adjustments to the DBS therapy for the identified patterns.

Example 33 includes subject matter that includes non-transitory machine-readable medium including instructions, which when executed by a machine, cause the machine to perform a method. The method performed by the machine may include delivering a deep brain stimulation (DBS) therapy to a patient according to a first therapy program, receiving a logged event from a user or sensor indicative of an acute reduction in therapy, automatically identifying a second therapy program for the DBS therapy to address the acute reduction in therapy, delivering the DBS therapy according to the second therapy program, prompting the user to report whether the user likes or dislikes the DBS therapy delivered according to the second therapy program, and controlling the DBS therapy based on the report. Controlling the DBS therapy based on the report may include choosing a third therapy program when the user does not like the second therapy program, reverting to the first therapy program when the user does not like the second therapy program, or continuing to deliver the DBS therapy according to the second therapy program when the user likes the second therapy program.

In further examples, the subject matter of Example 33 may be configured such that the method performed by the machine may include any of the subject matter recited in Examples 17-32.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an electrical stimulation system, which may be used to deliver DBS.

FIG. 2 illustrates, by way of example and not limitation, an implantable pulse generator (IPG) in a DBS system.

FIGS. 3A-3B illustrate, by way of example and not limitation, leads that may be coupled to the IPG to deliver electrostimulation such as DBS.

FIG. 4 illustrates, by way of example and not limitation, a computing device for programming or controlling the operation of an electrical stimulation system.

FIG. 5 illustrates, by way of example and not limitation, a more generalized example of a medical system that includes a medical device and a processing system.

FIG. 6 illustrates, by way of example, an example of an electrical therapy-delivery system.

FIG. 7 illustrates, by way of example and not limitation, a monitoring system and/or the electrical therapy-delivery system of FIG. 6, implemented using an IMD.

FIG. 8 illustrates, by way of example and not limitation, a method for addressing an acute reduction in therapy.

FIG. 9 illustrates, by way of example and not limitation, a system used for delivering a therapy such as a DBS therapy.

FIG. 10 illustrates, by way of example and not limitation, a method for creating a cumulative score for a plurality of clinical effect data (CED) which may be used to evaluate and compare therapy programs against each other.

FIG. 11 illustrates, by way of example and not limitation, a therapy space map.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

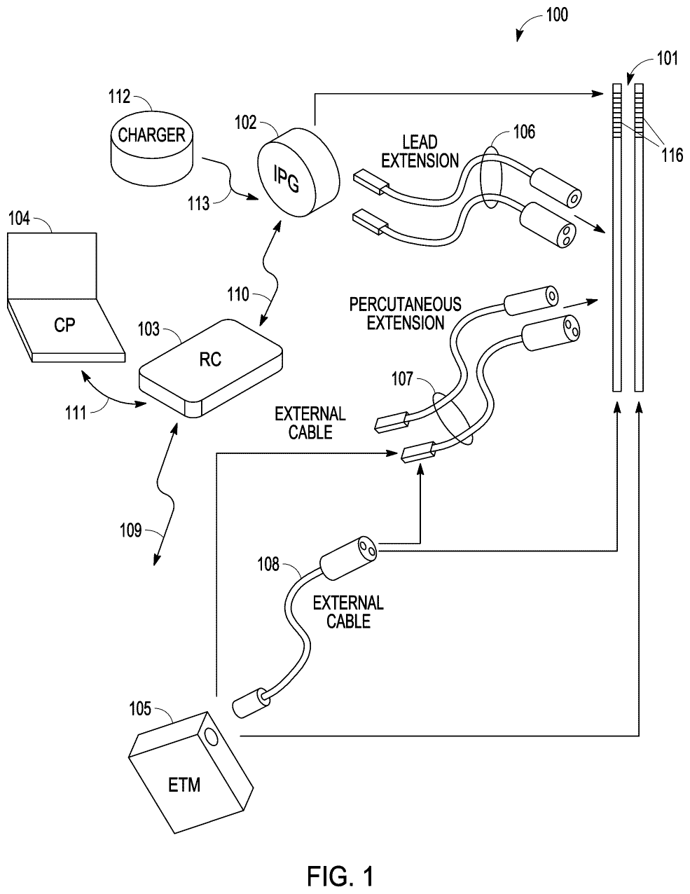

FIG. 1 illustrates, by way of example and not limitation, an electrical stimulation system 100, which may be used to deliver DBS. The electrical stimulation system 100 may generally include a one or more (illustrated as two) of implantable neuromodulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neuromodulation lead(s) 101, which carry a plurality of electrodes 116. The electrodes, when implanted in a patient, form an electrode arrangement. As illustrated, the neuromodulation leads 101 may be percutaneous leads with the electrodes arranged in-line along the neuromodulation leads or about a circumference of the neuromodulation leads. Any suitable number of neuromodulation leads can be provided, including only one, as long as the number of electrodes is greater than two (including the IPG case function as a case electrode) to allow for lateral steering of the current. Other types of leads may be used. The IPG 102 includes pulse generation circuitry that delivers electrical modulation energy in the form of a pulsed electrical waveform (i.e., a temporal series of electrical pulses) to the electrodes in accordance with a set of modulation parameters.

The ETM 105 may also be physically connected via the percutaneous lead extensions 107 and external cable 108 to the neuromodulation lead(s) 101. The ETM 105 may have similar pulse generation circuitry as the IPG 102 to deliver electrical modulation energy to the electrodes in accordance with a set of modulation parameters. The ETM 105 is a non-implantable device that may be used on a trial basis after the neuromodulation leads 101 have been implanted and prior to implantation of the IPG 102, to test the responsiveness of the modulation that is to be provided. Functions described herein with respect to the IPG 102 can likewise be performed with respect to the ETM 105.

The RC 103 may be used to telemetrically control the ETM 105 via a bi-directional RF communications link 109. The RC 103 may be used to telemetrically control the IPG 102 via a bi-directional RF communications link 110. Such control allows the IPG 102 to be turned on or off and to be programmed with different modulation parameter sets. The IPG 102 may also be operated to modify the programmed modulation parameters to actively control the characteristics of the electrical modulation energy output by the IPG 102. A clinician may use the CP 104 to program modulation parameters into the IPG 102 and ETM 105 in the operating room and in follow-up sessions.

The CP 104 may indirectly communicate with the IPG 102 or ETM 105, through the RC 103, via an IR communications link 111 or another link. The CP 104 may directly communicate with the IPG 102 or ETM 105 via an RF communications link or other link (not shown). The clinician detailed modulation parameters provided by the CP 104 may also be used to program the RC 103, so that the modulation parameters can be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 104. Alternatively, such programming methodologies can be performed using firmware or hardware. In any event, the CP 104 may actively control the characteristics of the electrical modulation generated by the IPG 102 to allow the desired parameters to be determined based on patient feedback or other feedback and for subsequently programming the IPG 102 with the desired modulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical modulation energy output by the neuromodulation leads, and select and program the IPG with modulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 112 may be a portable device used to transcutaneously charge the IPG 102 via a wireless link such as an inductive link 113. Once the IPG 102 has been programmed, and its power source has been charged by the external charger or otherwise replenished, the IPG 102 may function as programmed without the RC 103 or CP 104 being present.

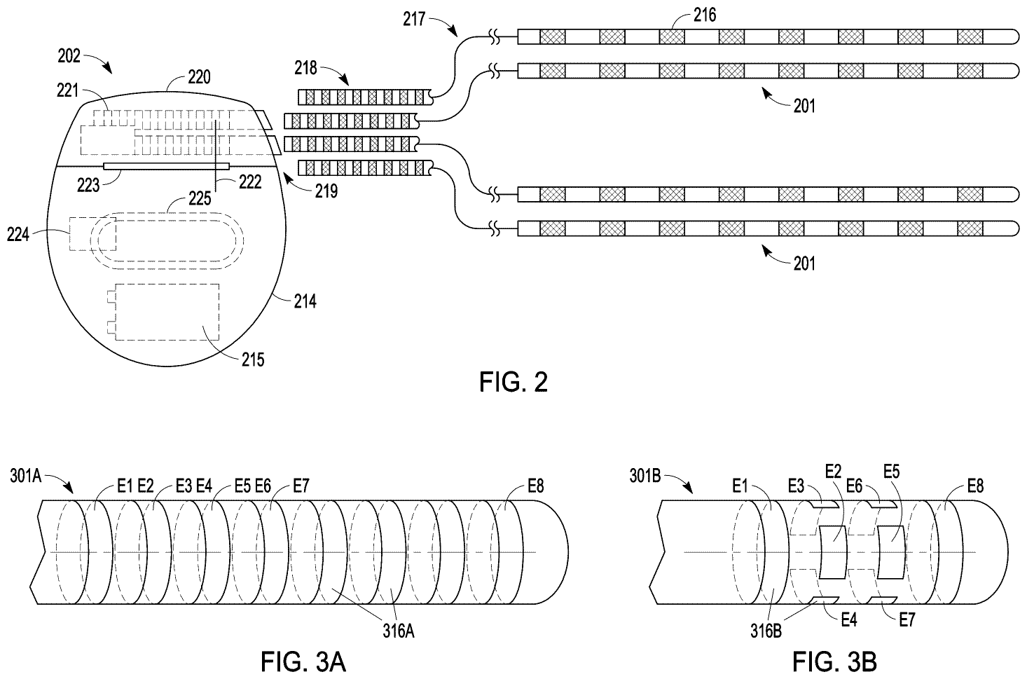

FIG. 2 illustrates, by way of example and not limitation, an IPG 202 in a DBS system. The IPG 202, which is an example of the IPG 102 of the electrical stimulation system 100 as illustrated in FIG. 1, may include a biocompatible device case 214 that holds the circuitry and a battery 215 for providing power for the IPG 202 to function, although the IPG 202 can also lack a battery and can be wirelessly powered by an external source. The IPG 202 may be coupled to one or more leads, such as leads 201 as illustrated herein. The leads 201 can each include a plurality of electrodes 216 for delivering electrostimulation energy, recording electrical signals, or both. In some examples, the leads 201 can be rotatable so that the electrodes 216 can be aligned with the target neurons after the neurons have been located such as based on the recorded signals. The electrodes 216 can include one or more ring electrodes, and/or one or more sets of segmented electrodes (or any other combination of electrodes), examples of which are discussed below with reference to FIGS. 3A and 3B.

The leads 201 can be implanted near or within the desired portion of the body to be stimulated. In an example of operations for DBS, access to the desired position in the brain can be accomplished by drilling a hole in the patient's skull or cranium with a cranial drill (commonly referred to as a burr), and coagulating and incising the dura mater, or brain covering. A lead can then be inserted into the cranium and brain tissue with the assistance of a stylet (not shown). The lead can be guided to the target location within the brain using, for example, a stereotactic frame and a microdrive motor system. In some examples, the microdrive motor system can be fully or partially automatic. The microdrive motor system may be configured to perform actions such as inserting, advancing, rotating, or retracing the lead.

Lead wires 217 within the leads may be coupled to the electrodes 216 and to proximal contacts 218 insertable into lead connectors 219 fixed in a header 220 on the IPG 202, which header can comprise an epoxy for example. Alternatively, the proximal contacts 218 may connect to lead extensions (not shown) which are in turn inserted into the lead connectors 219. Once inserted, the proximal contacts 218 connect to header contacts 221 within the lead connectors 219, which are in turn coupled by feedthrough pins 222 through a case feedthrough 223 to stimulation circuitry 224 within the case 214. The type and number of leads, and the number of electrodes, in an IPG is application specific and therefore can vary.

The IPG 202 can include an antenna 225 allowing it to communicate bi-directionally with a number of external devices. The antenna 225 may be a conductive coil within the case 214, although the coil of the antenna 225 may also appear in the header 220. When the antenna 225 is configured as a coil, communication with external devices may occur using near-field magnetic induction. The IPG 225 may also include a Radio-Frequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, Medical Implant Communication System (MICS), and the like.

In a DBS application, as is useful in the treatment of tremor in Parkinson's disease for example, the IPG 202 is typically implanted under the patient's clavicle (collarbone). The leads 201 (which may be extended by lead extensions, not shown) can be tunneled through and under the neck and the scalp, with the electrodes 216 implanted through holes drilled in the skull and positioned for example in the subthalamic nucleus (STN) and the pedunculopontine nucleus (PPN) in each brain hemisphere. The IPG 202 can also be implanted underneath the scalp closer to the location of the electrodes' implantation. The leads 201, or the extensions, can be integrated with and permanently connected to the IPG 202 in other solutions.

Stimulation in IPG 202 is typically provided by pulses each of which may include one phase or multiple phases. For example, a monopolar stimulation current can be delivered between a lead-based electrode (e.g., one of the electrodes 216) and a case electrode. A bipolar stimulation current can be delivered between two lead-based electrodes (e.g., two of the electrodes 216). Stimulation parameters typically include current amplitude (or voltage amplitude), frequency, pulse width of the pulses or of its individual phases; electrodes selected to provide the stimulation; polarity of such selected electrodes, i.e., whether they act as anodes that source current to the tissue, or cathodes that sink current from the tissue. Each of the electrodes can either be used (an active electrode) or unused (OFF). When the electrode is used, the electrode can be used as an anode or cathode and carry anodic or cathodic current. In some instances, an electrode might be an anode for a period of time and a cathode for a period of time. These and possibly other stimulation parameters taken together comprise a stimulation program that the stimulation circuitry 224 in the IPG 202 can execute to provide therapeutic stimulation to a patient.

In some examples, a measurement device coupled to the muscles or other tissue stimulated by the target neurons, or a unit responsive to the patient or clinician, can be coupled to the IPG 202 or microdrive motor system. The measurement device, user, or clinician can indicate a response by the target muscles or other tissue to the stimulation or recording electrode(s) to further identify the target neurons and facilitate positioning of the stimulation electrode(s). For example, if the target neurons are directed to a muscle experiencing tremors, a measurement device can be used to observe the muscle and indicate changes in, for example, tremor frequency or amplitude in response to stimulation of neurons. Alternatively, the patient or clinician can observe the muscle and provide feedback.

FIGS. 3A-3B illustrate, by way of example and not limitation, leads that may be coupled to the IPG to deliver electrostimulation such as DBS. FIG. 3A shows a lead 301A with electrodes 316A disposed at least partially about a circumference of the lead 301A. The electrodes 316A may be located along a distal end portion of the lead. As illustrated herein, the electrodes 316A are ring electrodes that span 360 degrees about a circumference of the lead 301. A ring electrode allows current to project equally in every direction from the position of the electrode, and typically does not enable stimulus current to be directed from only a particular angular position or a limited angular range around of the lead. A lead which includes only ring electrodes may be referred to as a non-directional lead.

FIG. 3B shows a lead 301B with electrodes 316B including ring electrodes such as E1 at a proximal end and E8 at the distal end. Additionally, the lead 301 also include a plurality of segmented electrodes (also known as split-ring electrodes). For example, a set of segmented electrodes E2, E3, and E4 are around the circumference at a longitudinal position, each spanning less than 360 degrees around the lead axis. In an example, each of electrodes E2, E3, and E4 spans 90 degrees, with each being separated from the others by gaps of 30 degrees. Another set of segmented electrodes E5, E6, and E7 are located around the circumference at another longitudinal position different from the segmented electrodes E2, E3 and E4. Segmented electrodes such as E2-E7 can direct stimulus current to a selected angular range around the lead.

Segmented electrodes can typically provide superior current steering than ring electrodes because target structures in DBS or other stimulation are not typically symmetric about the axis of the distal electrode array. Instead, a target may be located on one side of a plane running through the axis of the lead. Through the use of a radially segmented electrode array, current steering can be performed not only along a length of the lead but also around a circumference of the lead. This provides precise three-dimensional targeting and delivery of the current stimulus to neural target tissue, while potentially avoiding stimulation of other tissue. In some examples, segmented electrodes can be together with ring electrodes. A lead which includes at least one or more segmented electrodes may be referred to as a directional lead. In an example, all electrodes on a directional lead can be segmented electrodes. In another example, there can be different numbers of segmented electrodes at different longitudinal positions.

Segmented electrodes may be grouped into sets of segmented electrodes, where each set is disposed around a circumference at a particular longitudinal location of the directional lead. The directional lead may have any number of segmented electrodes in a given set of segmented electrodes. By way of example and not limitation, a given set may include any number between two to sixteen segmented electrodes. In an example, all sets of segmented electrodes may contain the same number of segmented electrodes. In another example, one set of the segmented electrodes may include a different number of electrodes than at least one other set of segmented electrodes.

The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The sets of segmented electrodes may be positioned in irregular or regular intervals along a length the lead 219.

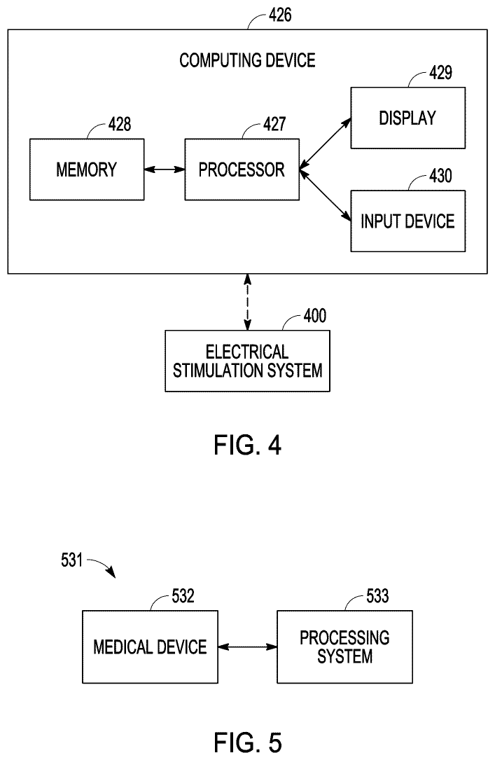

FIG. 4 illustrates, by way of example and not limitation, a computing device 426 for programming or controlling the operation of an electrical stimulation system 400. The computing device 426 may include a processor 427, a memory 428, a display 429, and an input device 430. Optionally, the computing device 426 may be separate from and communicatively coupled to the electrical stimulation system 400, such as system 100 in FIG. 1. Alternatively, the computing device 426 may be integrated with the electrical stimulation system 100, such as part of the IPG 102, RC 103, CP 104, or ETM 105 illustrated in FIG. 1.

The computing device 426, also referred to as a programming device, can be a computer, tablet, mobile device, or any other suitable device for processing information. The computing device 426 can be local to the user or can include components that are non-local to the computer including one or both of the processor 427 or memory 428 (or portions thereof). For example, the user may operate a terminal that is connected to a non-local processor or memory. The functions associated with the computing device 426 may be distributed among two or more devices, such that there may be two or more memory devices performing memory functions, two or more processors performing processing functions, two or more displays performing display functions, and/or two or more input devices performing input functions. In some examples, the computing device 406 can include a watch, wristband, smartphone, or the like. Such computing devices can wirelessly communicate with the other components of the electrical stimulation system, such as the CP 104, RC 103, ETM 105, or IPG 102 illustrated in FIG. 1. The computing device 426 may be used for gathering patient information, such as general activity level or present queries or tests to the patient to identify or score pain, depression, stimulation effects or side effects, cognitive ability, or the like. In some examples, the computing device 426 may prompt the patient to take a periodic test (for example, every day) for cognitive ability to monitor, for example, Alzheimer's disease. In some examples, the computing device 426 may detect, or otherwise receive as input, patient clinical responses to electrostimulation such as DBS, and determine or update stimulation parameters using a closed-loop algorithm based on the patient clinical responses. Examples of the patient clinical responses may include physiological signals (e.g., heart rate) or motor parameters (e.g., tremor, rigidity, bradykinesia). The computing device 426 may communicate with the CP 104, RC 103, ETM 105, or IPG 102 and direct the changes to the stimulation parameters to one or more of those devices. In some examples, the computing device 426 can be a wearable device used by the patient only during programming sessions. Alternatively, the computing device 426 can be worn all the time and continually or periodically adjust the stimulation parameters. In an example, a closed-loop algorithm for determining or updating stimulation parameters can be implemented in a mobile device, such as a smartphone, that is connected to the IPG or an evaluating device (e.g., a wristband or watch). These devices can also record and send information to the clinician.

The processor 427 may include one or more processors that may be local to the user or non-local to the user or other components of the computing device 426. A stimulation setting (e.g., parameter set) includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. The stimulation parameters may include, for example, current amplitude values, current fractionalization across electrodes, stimulation frequency, stimulation pulse width, etc.

The processor 427 may identify or modify a stimulation setting through an optimization process until a search criterion is satisfied, such as until an optimal, desired, or acceptable patient clinical response is achieved. Electrostimulation programmed with a setting may be delivered to the patient, clinical effects (including therapeutic effects and/or side effects, or motor symptoms such as bradykinesia, tremor, or rigidity) may be detected, and a clinical response may be evaluated based on the detected clinical effects. When actual electrostimulation is administered, the settings may be referred to as tested settings, and the clinical responses may be referred to as tested clinical responses. In contrast, for a setting in which no electrostimulation is delivered to the patient, clinical effects may be predicted using a computational model based at least on the clinical effects detected from the tested settings, and a clinical response may be estimated using the predicted clinical effects. When no electrostimulation is delivered the settings may be referred to as predicted or estimated settings, and the clinical responses may be referred to as predicted or estimated clinical responses.

In various examples, portions of the functions of the processor 427 may be implemented as a part of a microprocessor circuit. The microprocessor circuit can be a dedicated processor such as a digital signal processor, application specific integrated circuit (ASIC), microprocessor, or other type of processor for processing information. Alternatively, the microprocessor circuit can be a processor that can receive and execute a set of instructions of performing the functions, methods, or techniques described herein.

The memory 428 can store instructions executable by the processor 427 to perform various functions including, for example, determining a reduced or restricted electrode configuration and parameter search space (also referred to as a “restricted search space”), creating or modifying one or more stimulation settings within the restricted search space, etc. The memory 428 may store the search space, the stimulation settings including the “tested” stimulation settings and the “predicted” or “estimated” stimulation settings, clinical effects (e.g., therapeutic effects and/or side effects) and clinical responses for the settings.

The memory 428 may be a computer-readable storage media that includes, for example, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information, and which can be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, Bluetooth, near field communication, and other wireless media.

The display 429 may be any suitable display or presentation device, such as a monitor, screen, display, or the like, and can include a printer. The display 429 may be a part of a user interface configured to display information about stimulation settings (e.g., electrode configurations and stimulation parameter values and value ranges) and user control elements for programming a stimulation setting into an IPG.

The input device 430 may be, for example, a keyboard, mouse, touch screen, track ball, joystick, voice recognition system, or any combination thereof, or the like. Another input device 430 may be a camera from which the clinician can observe the patient. Yet another input device 430 may a microphone where the patient or clinician can provide responses or queries.

The electrical stimulation system 400 may include, for example, any of the components illustrated in FIG. 1. The electrical stimulation system 400 may communicate with the computing device 426 through a wired or wireless connection or, alternatively or additionally, a user can provide information between the electrical stimulation system 400 and the computing device 426 using a computer-readable medium or by some other mechanism.

FIG. 5 illustrates, by way of example and not limitation, a more generalized example of a medical system 531 that includes a medical device 532 and a processing system 533. For example, the electrical stimulation system 400 of FIG. 4 may be a more specific example of the medical device 532 of FIG. 5, and computing device 426 of FIG. 4 may be a more specific example of the processing system 533 of FIG. 5. The medical device may be configured to provide sensing functions and/or therapy functions. For example, the medical device may include a device configured to use a parameter set to deliver an electrical stimulation therapy. The medical device may be an implantable medical device such as an implantable neurostimulator. The implantable medical device may be configured to deliver SCS or DBS therapy. The medical device may include more than one medical device. The processing system may be within a single device, or may be a distributed system across two or more devices including local and/or remote systems. According to various embodiments, the medical system may include at least one medical device configured to treat a condition by delivering a therapy to a patient.

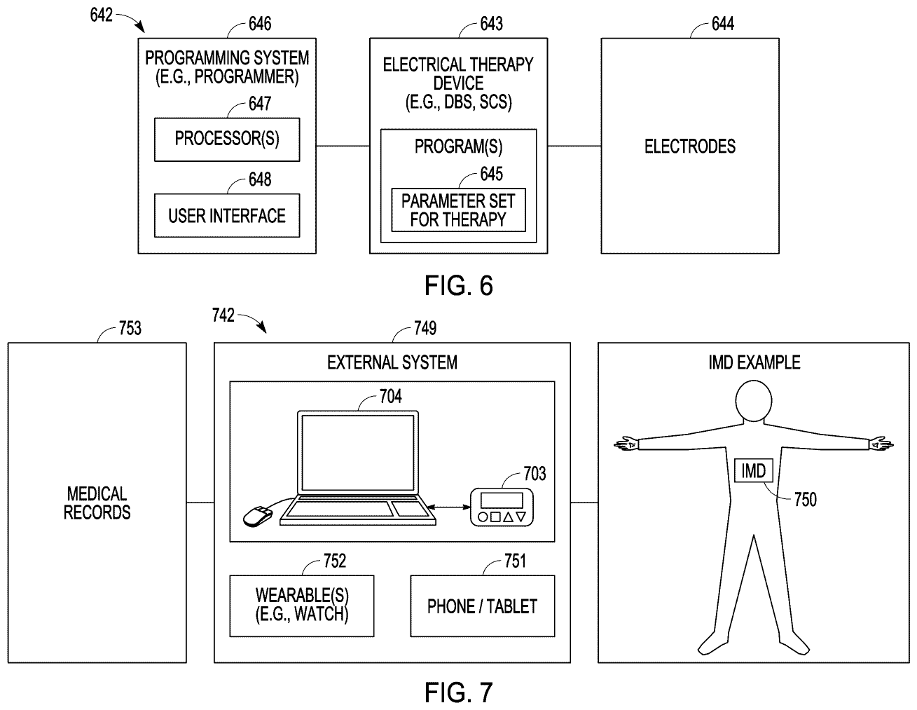

FIG. 6 illustrates, by way of example, an example of an electrical therapy-delivery system. The illustrated system 642 may be a more specific example of the system illustrated in FIG. 5, or form a portion of the system illustrated in FIG. 5. The illustrated system 642 includes an electrical therapy device 643 configured to deliver an electrical therapy to electrodes 644 to treat a condition in accordance with a programmed parameter set 645 for the therapy. The system 642 may include a programming system 646, which may function as at least a portion of a processing system, that may include one or more processors 647 and a user interface 648. The programming system 646 may be used to program and/or evaluate the parameter set(s) used to deliver the therapy. The illustrated system 642 may be a DBS system.

In some embodiments, the illustrated system 642 may include an SCS system to treat pain and/or a system for monitoring pain. By way of example, a therapeutic goal for conventional SCS programming may be to maximize stimulation (i.e., recruitment) of the dorsal column (DC) fibers that run in the white matter along the longitudinal axis of the spinal cord and minimal stimulation of other fibers that run perpendicular to the longitudinal axis of the spinal cord (e.g., dorsal root fibers).

A therapy may be delivered according to a parameter set. The parameter set may be programmed into the device to deliver the specific therapy using specific values for a plurality of therapy parameters. For example, the therapy parameters that control the therapy may include pulse amplitude, pulse frequency, pulse width, and electrode configuration (e.g., selected electrodes, polarity and fractionalization). The parameter set includes specific values for the therapy parameters. The number of electrodes available combined with the ability to generate a variety of complex electrical waveforms (e.g., pulses), presents a huge selection of modulation parameter sets to the clinician or patient. For example, if the neuromodulation system to be programmed has sixteen electrodes, millions of modulation parameter sets may be available for programming into the neuromodulation system. To facilitate such selection, the clinician generally programs the modulation parameters sets through a computerized programming system to allow the optimum modulation parameters to be determined based on patient feedback or other means and to subsequently program the desired modulation parameter sets.

FIG. 7 illustrates, by way of example and not limitation, the electrical therapy-delivery system of FIG. 6 implemented using an IMD. The IMD may include a DBS stimulator. The illustrated system 742 includes an external system 749 that may include at least one programming device. The illustrated external system 749 may include a clinician programmer 704, similar to CP 104 in FIG. 1, configured for use by a clinician to communicate with and program the neuromodulator, and a remote control device 703, similar to RC 103 in FIG. 1, configured for use by the patient to communicate with and program the neuromodulator. For example, the remote control device 703 may allow the patient to turn a therapy on and off, change or select programs, and/or may allow the patient to adjust patient-programmable parameter(s) of the plurality of modulation parameters. FIG. 7 illustrates an IMD 750, although the monitor and/or therapy device may be an external device such as a wearable device. The external system 749 may include a network of computers, including computer(s) remotely located from the IMD 750 that are capable of communicating via one or more communication networks with the programmer 704 and/or the remote control device 703. The remotely located computer(s) and the IMD 750 may be configured to communicate with each other via another external device such as the programmer 704 or the remote control device 703. The remote control device 703 and/or the programmer 704 may allow a user (e.g., patient, caregiver and/or clinician or rep) to answer questions as part of a data collection process. The external system 749 may include personal devices such as a phone or tablet 751, wearables such as a watch 752, sensors or therapy-applying devices. The watch may include sensor(s), such as sensor(s) for detecting activity, motion and/or posture. Other wearable sensor(s) may be configured for use to detect activity, motion and/or posture of the patient. The external system 749 may include, but is not limited to, a phone and/or a tablet. The system 742 may include medical record(s) 753 for the patient and broader patient population(s). The medical record(s) may be stored and accessed using one or more servers (e.g., local or remote servers such as cloud-based servers).

Embodiments of the present subject matter provide improved systems for counteracting an acute reduction in therapeutic outcome. Some embodiments provide a system capable of using user logging, such as logs entered by a patient and/or caregiver, to respond to acute reductions in therapy, identify patterns of therapy reduction, and develop novel event related adjustments for patients. The feedback may include current symptoms, side effects and patient activities to adjust weighting for symptoms/side effects and select a stimulation program.

Systems may include a mobile DBS application enabling user interaction which may be used to receive user logs and to provide stimulation suggestions. The system may be configured to upload or otherwise identify the current stimulation settings as well as to provide alternative stimulation options. The system may be configured to automatically push stimulation recommendations by directly making changes to the patient's stimulator.

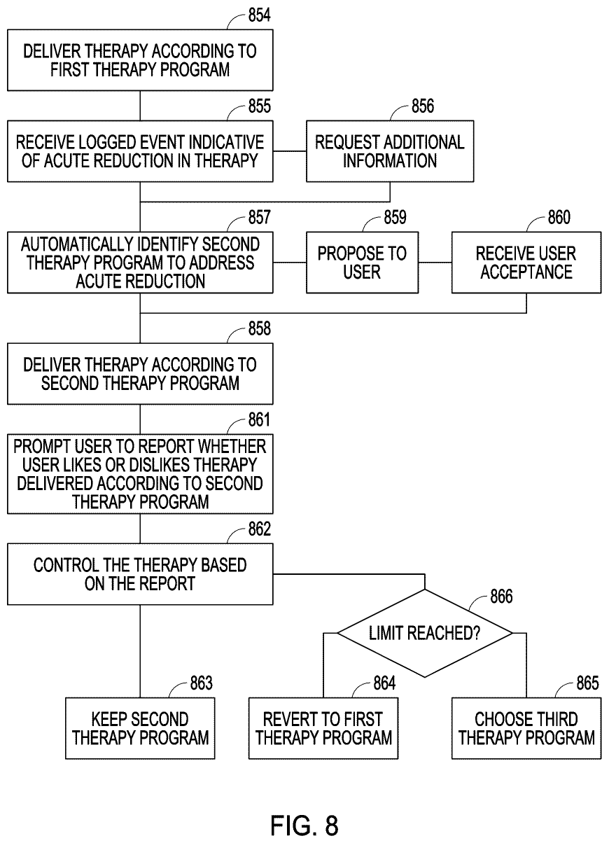

FIG. 8 illustrates, by way of example and not limitation, a method for addressing an acute reduction in therapy. The illustrated method includes delivering a therapy to a patient according to a first therapy program 854. The therapy may be, by way of example and not limitation, a deep brain stimulation (DBS) therapy. At 855, the system receives a logged event indicative of an acute reduction in therapy. The event may be logged via user input (e.g., patient and/or caregiver), and/or the event may be logged using sensor(s). Other data source(s) may be used to determine the event. For example, the other data source(s) may be used to provide contextual information for the user-logged event. For some event(s), as illustrated at 856, the system may be configured to request additional information from the user and/or receive additional information from database(s) and/or sensor(s). The illustrated system may be configured to automatically identify a second therapy program for the therapy (e.g., DBS therapy) to address the acute reduction 857. The system may be configured to deliver the therapy (e.g., DBS therapy) according to the identified second therapy program 858 to address the acute reduction in therapy. In some embodiments, the therapy is automatically delivered after identifying the second therapy program. According to some embodiments, the system may propose the identified second therapy program to the user 859 and receive acceptance from the user 860 before delivering the therapy according to the second therapy program 858 to address the acute reduction in therapy. At 861, the system is configured to prompt the user to report whether the user likes or dislikes the DBS therapy delivered according to the second therapy program. One or more of these prompts may be delivered after one or more corresponding times after the therapy is delivered. It is noted that some embodiments of the system may be configured to automatically detect that the treatment is ineffective or is causing an undesirable outcome and may be configured to automatically revert to the previous or original program or to a predefined “safe program.” Such systems may avoid the “prompt” at 861. At 862, the system may be configured to control the therapy based on the report. By way of example and not limitation, the therapy may be controlled by keeping the second therapy program 863 such as may be appropriate when the patient likes or is satisfied with the therapy. For example, the patient may indicate that the therapy is “good.” If the patient does not like the second therapy program, the system may revert from the second therapy program to the first therapy program 864 or may choose another therapy program (e.g., third therapy program) 865. Some embodiments may check for whether a limit for switching programs has been reached 866 before switching programs. This limit may be used to prevent a user from switching programs too often or too quickly before the program even washes in.

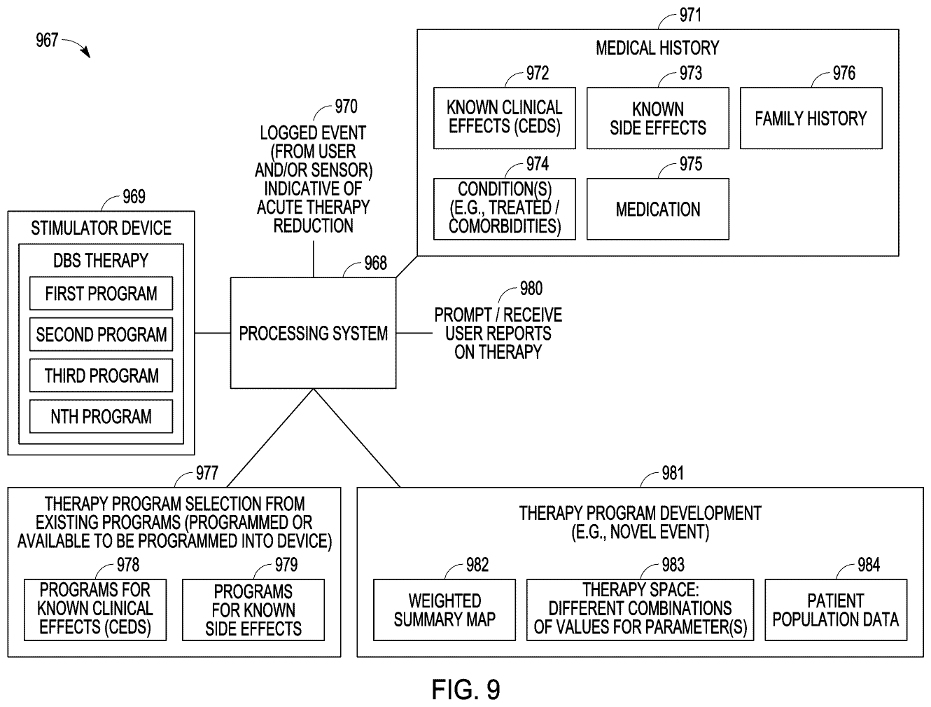

FIG. 9 illustrates, by way of example and not limitation, a system used for delivering a therapy such as a DBS therapy. The illustrated system 967 includes a processing system 968, which may be in a single device or may be distributed across more than one device, operably connected to a stimulator device 969 that is configured to deliver a therapy. The stimulator device 969 may include a DBS stimulator configured to deliver a DBS therapy. The stimulator device may be programmed to deliver the therapy according to programs stored within the programmer. For example, the illustrated stimulator device 969 includes a first program, a second program, a third program, and may include more program(s) (e.g., Nth program). As discussed in more detail below, the processing system may be configured to determine a program recommendation from existing programs, create new programs or modify existing programs to create different programs for storage in the stimulator device.

The processing system 968 may be configured to receive a logged event indicative of an acute therapy reduction 970. The user (e.g., patient and/or caregiver) may log the event via user input into the processing system and/or a sensor or sensors may be used to sense parameter(s) used to detect the event indicative of the acute therapy reduction.

The processing system 968 may be configured to provide additional context for the logged event by accessing a medical history 971 for the patient. By way of example and not limitation, the processing system 968 may access one or more of known clinical effect data (CED) 972 for the patient, known side effect(s) 973 that the patient may experience, medical condition(s) 974 for the patient such as comorbidities that may contribute the acute therapy reduction, medication information 975 such as medicine, dose and schedule that the patient takes, and family history information 976 which may also provide insight into the acute therapy reduction by, for example, identifying undiagnosed medical condition(s) that may contribute to the acute therapy reduction. Medical history may include data collected in the ambulatory patient and uploaded to cloud-based servers for review by clinicians. Examples of such data may include, but are not limited to, weight, cardiovascular data such as blood pressure, heart rate and heart rhythm, blood glucose numbers, diet, and the like which may be collected to monitor and/or treat conditions such as, but not limited to, a heart disease or diabetes. Thus, by way of example, an acute therapy reduction may be determined to be at least partially attributable to blood glucose that is out of range or a cardiac arrhythmic episode. By way of another example, an acute therapy reduction for a movement disorder therapy may be determined to be at least partially attributable to the patient's medication state. For example, when DBS patients are treated with both medication and stimulation to manage their motor symptoms, the patient's motor symptoms can fluctuate depending on where they are in their medication cycle by generally increase as a dose is absorbed and decreasing with drug clearance through metabolism and elimination.

The processing system 968 may respond to the logged event by selecting a therapy program from a set of existing therapy programs that are already programmed in the stimulator device 969 or are already created and available to be programmed into the stimulator device. Thus, by way of example, the system may determine a program for delivering the therapy using the second program rather than the first program. The program(s) 977 available for selection may be determined to be useful for known clinical effect(s) 978 and/or may be determined to be useful for known side effects 979. The processing system 938 may use a user interface to send a prompt 980 to the user (e.g., patient and/or caregiver) requesting whether the user would like to use the recommended program.

The processing system 968 may respond to the logged event by developing a therapy program, such as may be useful to address a novel event. The therapy program development 981 may use a weighted summary map 982, a therapy space 983 representing various combinations of values for parameter(s) that may be searched to find a therapeutically effective program for the novel event, and may use patient population data 984 to determine the program from medical records for other patients.

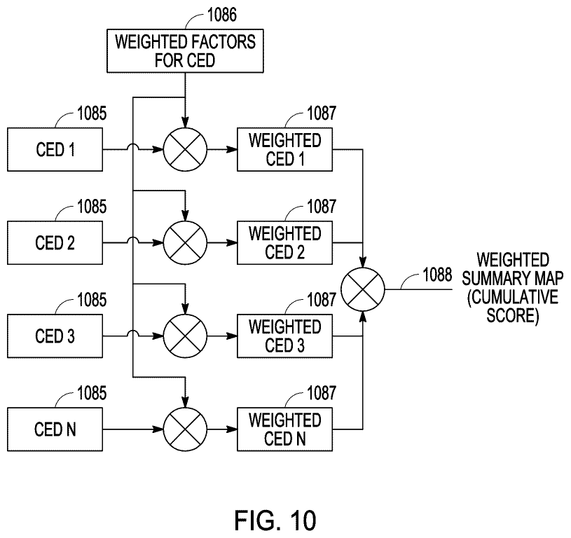

FIG. 10 illustrates, by way of example and not limitation, a method for creating a cumulative score for a plurality of clinical effect data (CED) which may be used to evaluate and compare therapy programs against each other. One or more CEDs 1085 (e.g., CED 1-N) may be identified, and weight factors 1086 may be applied to each of the CEDs to provide one or more weighted CEDs 1087 (e.g., Weighted CEDs 1-N). The weighted CEDs 1087 may be summed together to provide a cumulative score 1088 or weighted summary map for the program. The cumulative score may be compared to cumulative scores for other therapies to determine which program to recommend for use to deliver the therapy.

Various embodiments respond to the following three logged scenarios. In the first scenario, the user (e.g., patient and/or caregiver) knows the symptom(s) that has acute reduction in therapy. In the second scenario, the user (e.g., patient and/or caregiver) knows stimulation induced side effect that is occurring. In the third scenario, the user (e.g., patient and/or caregiver) logs an acute reduction in therapy coupled to an activity that may involve a complex set of symptoms. If the scenario is repeated and the appropriate response is predictable, the process may be altered to reduce required user interaction to address the scenario.

The user (e.g., patient or caregiver) knows that the patient has systems and has an acute reduction in relief of these symptoms in the first scenario. The workflow may be initiated when a user (e.g., patient and/or caregiver) logs an acute reduction in therapy for a given symptom. The workflow may also be initiated if a sensor or the system is capable of detecting the symptom deterioration.

For example, the user may log an event (e.g., log a tremor event). The system may be configured to identify when the logged event is significant compared to previous logged events, and may further be configured to identify an accessible stimulation program that is expected to better target the significant event. For the logged tremor event example, the system may be configured to identify that the patient is experiencing a tremor increase that is significant compared to the patient's average tremor response, and may further be configured to identify an accessible stimulation program that should better target the tremor system (based on stored historical data such as clinical effects data (CED), patient reported data, wearable recordings, and the like).

The system may be configured to propose the alternative stimulation program to the user. If the user accepts, the system will implement the alternative program. Some system embodiments may be configured to automatically detect an impact of the change on the patient and make a recommendation or automated change without prompting the patient for feedback. Some system embodiments include the user prompt. For example, after a first short time period after beginning the alternative program, the user may be prompted to report whether the symptom associated with the event (e.g., the tremor) is improved and whether they would like to continue using the new program. For example, the first time period may be less than a minute, less than 45 seconds, or less than 30 seconds after initiating the alternative stimulation program. It is expected that positive results for addressing some symptoms should appear very quickly because the symptom has a quick wash-in time. Thus, by prompting within the first time period, the system can quickly ascertain when the user knows that the alternatively stimulation program is not satisfactory. If the user (e.g., patient and/or caregiver) does not like the program, the user can choose to try another program (if available) or revert to the original program. A limit may be set on the number of alternative programs the user can try. For example, some users may be obsessive in continuing to toggle among programs with little improvement in the delivered therapy. This limit may be used to prevent users from switching programs before the therapy associated with programs can effectively wash-in. When the user chooses to continue with the therapy at the first time period, the user may be prompted again after a second time period after implementing the alternative program, longer than the first time period. This second prompt inquires if the user still likes the program. For example, the second time period may be between 15 minutes to two hours, may be between 20 minutes to 1 hour, or may be between 25 minutes to 35 minutes (e.g., about 30 minutes). The prompt at the second period can be used to consider the therapy's effect on a different symptom or side effect which may have a longer wash-in time. Yet at least one other prompt (e.g., third prompt) may be provided after a day or two, which may allow the system to account for longer wash-in times for some psychiatric symptoms or side effects with longer wash-in times. The addition third prompt (or more) may be added based on the user log. For example, the log entries may indicate that the patient is having an emotional side effect that may tend to have longer wash-in times. In another example, the user logs may indicate that patient has all of a sudden developed compulsive behavior such as compulsive gambling. The prompts given to the patient (or caregiver for situations in which the patient cannot self-identify) may correspond to desirable prompt times to intervene early in the therapy. The time period(s) may depend on a type of event identified by the user log. In an example where the symptom is measurable, such as measured using a wearable or internal sensor (e.g., accelerometer), a mobile task, a voice task, and the like, the measurable symptom may be used in alone or in combination with the user log (e.g., user-provided feedback) to inform the system of the outcome of the treatment.

The system may be configured to look for new symptoms that are known to develop as the patient's condition changes. For example, additional motor problems may develop as Parkinson's Disease progresses and the system may be configured to consider whether the patient is experiencing one of these conditions, and then apply a therapy program known to be effective in alleviating that condition. This therapy program may have been developed from a broader patient population.

The second scenario is where the user (e.g., patient or caregiver) knows that the patient may experience a stimulation-induced side effect, and that an acute reduction in therapy increases the undesired side effect of the stimulation. The workflow may be initiated when a user (e.g., patient and/or caregiver) logs the presence of a known side effect which the user desired to reduce. The workflow may also be initiated if a sensor or the system is capable of detecting the side effect. Similarly, the system may detect a symptom relief (e.g., relief from tremor) and/or may detect an unanticipated response from other detectable responses that are not traditionally considered to be symptoms or side effects, such as but not limited to heart rate or blood pressure.

For example, the user may log a side effect and the system may be configured to identify an accessible program that is expected to better avoid the side effect. For example, the patient may log a speech side effect and the system may be configured to identify an accessible stimulation program that should reduce the speech side effect while minimizing impact to therapy (based on stored historical data such as clinical effects data (CED), patient reported data, wearable recordings, and the like).

The system be configured to propose the alternative stimulation program to the user. If the user accepts, the system will implement the alternative program. After a first short time period after beginning the alternative program, the user may be prompted to report whether the side effect (e.g., speech) is improved and whether they would like to continue using the new program. For example, the first time period may be less than a minute, less than 45 seconds, or less than 30 seconds after initiating the alternative stimulation program. It is expected that positive results for addressing some symptoms should appears very quickly (quick wash-in time). Thus, the system can quickly ascertain when the user knows that the alternatively stimulation program is not satisfactory. If the user (e.g., patient and/or caregiver) does not like the program, the user can choose to try another program (if available) or revert to the original program. A limit may be set on the number of alternative programs the user can try. For example, some users may be obsessive in continuing to toggle among programs with little improvement in the delivered therapy. After a second time period after implementing the alternative program, longer than the first time period, the user may be prompted again if the user still likes the program. For example, the second time period may be between 15 minutes to two hours, may be between 20 minutes to 1 hour, or may be between 25 minutes to 35 minutes (e.g., about 30 minutes). When the user chooses to continue with the therapy at the first time period, the user may be prompted again after a second time period after implementing the alternative program, longer than the first time period. This second prompt inquires if the user still likes the program. For example, the second time period may be between 15 minutes to two hours, may be between 20 minutes to 1 hour, or may be between 25 minutes to 35 minutes (e.g., about 30 minutes). The prompt at the second period can be used to consider the therapy's effect on a different symptom or side effect which may have a longer wash-in time. Another prompt (e.g., third prompt) may be provided after a day or two, which may allow the system to account for longer wash-in times for some psychiatric symptoms or side effects with longer wash-in times. In an example where the symptom is measurable, such as measured using a wearable or internal sensor (e.g., accelerometer), a mobile task, a voice task, and the like, the measurable symptom may be used in alone or in combination with the user log (e.g., user-provided feedback) to inform the system of the outcome of the treatment. Some embodiments may change the time for prompting the user based on the type of side effect. For example, a psychological or emotional side effect may cause longer first and/r second time periods for prompting the user response. Additionally or alternatively, the additional time period(s) may be implemented to trigger additional prompts for receiving user feedback.

The third scenario is where the user (e.g., patient or caregiver) logs an acute reduction in therapy coupled to an activity that may involve a complex set of symptoms. For example, there could be a very complex combination of a loss of symptom relief or introduced side effects, medications, life events, and patient health. The patient or caregiver may know that something happened to cause the reduction in therapy, but they are having difficulty in determining how to fix it. The workflow may be initiated when a user (e.g., patient and/or caregiver) logs an activity where therapy is suboptimal. For example, the patient may log that therapy is suboptimal when eating dinner, but may otherwise be fine during other times including eating other meals. The system may query the patient about the activity to identify what symptoms and side effects may be contributing to the acute reduction in the therapy. The queries may be directed toward asking about specific symptoms or side effects directly (focusing on those the patient is known to have). The queries may be event specific and may focus on what difficulties might be associated with the given task. For the example where the patient has problems eating dinner, examples of queries may include: “Do you have difficulty using utensils?” “Do you have more difficulty using a spoon or a fork?” and “Are you having difficulty swallowing?”.

The system may be configured to impose time stamps on sensed inputs or user inputs. For example, the system may use the time stamps to detect subthreshold trends that occur during the identified event and may signal the involvement of specific symptoms, side effects, or other detectable change in health during a specified time as identified by the time stamps.

In addition or alternative to using queries, the system may be configured to receive free text from the user defining the difficulties and may use language processing to analyze the submitted text. Either based on the user's entries, or (if user entries are inconclusive) based on available alternative programs, the system may identify an accessible stimulation program predicted to improve therapy (based on stored historical data such as CED, patient reported data, wearable recordings, etc.).

The system be configured to propose the alternative stimulation program to the user. If the user accepts, the system will implement the alternative program. After a first short time period after beginning the alternative program, the user may be prompted to report whether the therapy improved (e.g., improvements in symptoms/side effects) and whether the user would like to continue using the new program. For example, the first time period may be less than a minute, less than 45 seconds, or less than 30 seconds after initiating the alternative stimulation program. Thus, the system can quickly ascertain when the user knows that the alternatively stimulation program is not satisfactory. If the user (e.g., patient and/or caregiver) does not like the program, the user can choose to try another program (if available) or revert to the original program. A limit may be set on the number of alternative programs the user can try. For example, some users may be obsessive in continuing to toggle among programs with little improvement in the delivered therapy. After a second time period after implementing the alternative program, longer than the first time period, the user may be prompted again if the user still likes the program. For example, the second time period may be between 15 minutes to two hours, may be between 20 minutes to 1 hour, or may be between 25 minutes to 35 minutes (e.g., about 30 minutes). In an example where the symptom is measurable, such as measured using a wearable or internal sensor (e.g., accelerometer), a mobile task, a voice task, and the like, the measurable symptom may be used in alone or in combination with the user log (e.g., user-provided feedback) to inform the system of the outcome of the treatment.

Alternative program may be selected by altering the weights or side effect regions of previously collected clinical effects data (CED). The weight of symptoms may initially be the weights set in clinic. If no weights were set, then all symptoms are weighted equally. A weighted summary map may be used to combine the weighting information into a cumulative score. If a symptom becomes worse, the weight of that symptom may be increased relative to the remaining symptoms, and the cumulative map may be updated based on the new weighting. The system may be configured to suggest the program using the program with the best estimated score remaining. It may be possible for two or more programs to have the same score. By way of example and not limitation, when programs have the same score, then the suggested program may be based on the program that was most recently tested, the distance from the side effects, and/or, the program with the less energy consumption. If a side effect becomes worse, a side effect barrier may be placed on the cumulative map without altering weights. The suggested program may be the one with the best estimated score remaining outside of a side effect region.

For example, a patient's symptoms and side effects may be evaluated, and a program may be selected to target particular symptoms and side effects. By way of example, a device may be programmed to prioritize tremor a little more than bradykinesia. However, when a patient attempts to cat, it may then be determined that the patient's bradykinesia is impacting them more than tremor. The weighting for treating bradykinesia may be increased for a program used to deliver therapy while the patient eats. This weighing changes the “hot spot” in the map for identifying effective parameters sets to be used in the program.

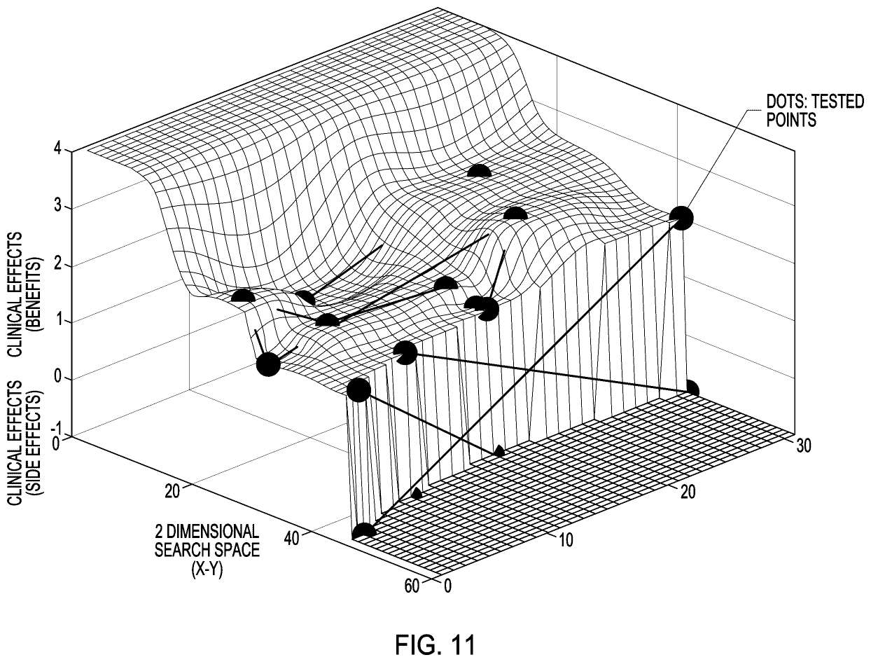

FIG. 11 illustrates, by way of example and not limitation, a therapy space map. The map includes tested points (“dots”) and estimated points (other points in map). For ease in visualizing a three-dimensional therapy space map, the search space has two parameters to provide an X-Y plane. More particularly, by way of example, the X-Y plane in the illustration represents pulse amplitude (0-60 axis) and electrode (0-30). The Z-axis (height) represents clinical effects, where positive values are benefits and negative values are side effects. It is noted that the search space may include many more parameters than two parameters. There often is a fairly-well defined delineation between the beneficial CEDS and the side effects (as shown in the illustrated map near about 40-55 one the pulse amplitude axis). The region where the parameter combinations provide beneficial clinical effects may be deemed to be the therapy search space. Where different parameter combinations provide similar clinical effects, the distance from this side effect boundary may be used in some examples to determine the therapy program recommendation. A weighted summary map combines weights of clinical effects data into a cumulative score. The replacement therapy program may be automatically identified by altering the weights of symptoms in the clinical effects data, and identifying a program with a best estimated score. The replacement therapy program may be automatically identified by placing a side effect barrier on the cumulative map. The automatically identified second therapy program may have a best estimated score remaining outside of a side effect region.

The therapy space map may be stored in the device to allow the device to search for other parameters sets within a safe search space. Thus, the device is not limited only to pre-programmed therapies in the device. Further, the system is not limited to pre-defined programs in external servers (e.g., cloud-based servers) that are available for use to program the stimulator device.

The present subject matter may recommend program(s) based on acute changes and may use newly collected clinical effects to alter the weights (or prioritization) of the symptom, calculate a new clinical effects map, and determine the best region to stimulate from the new map.

If any of these scenarios for the same symptom, same side effect or same activity are repeated and the appropriate response is predictable, the process may be altered to reduce required user interaction to address the scenario. The workflow may be altered such that the patient will not be re-queried about the efficacy of the program or for side effects if a working program was previously identified. For a working program related to an activity, the patient may be queried if they wish for the program to return to the main program after the activity. If yes, the user may be asked for the approximate duration of the activity. If yes, the user will define the duration and the program will revert at the scheduled time. If a user regularly repeats any of the above scenarios for the same symptom, side effect or activity, then the program may be stored as a permanent alternative program that may be easily accessed from a main screen or even via a stored trigger mechanism.

The patient may have multiple (e.g., four or more) different stored programs that may be easily accessed or secondary trigger such as, but not limited to a button press on a mobile device, a tap on an IPG, a button press on affiliated wearable, and the like. By way of example but not limitation, the stored programs may include a main program (most common), a high tremor program, a speech improvement program, and a program for eating dinner.

If an event regularly repeats around the same time on predictable days, the system may propose to the user a schedule to automatically activate/deactivate the alternative program(s) during given times of the day. Any auto activation may be accompanied by a notification to the user such as a sound, haptic feedback, or other notification mechanism.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encrypted with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method, comprising:
delivering a deep brain stimulation (DBS) therapy to a patient according to a first therapy program;
receiving a logged event from a user or sensor indicative of an acute reduction in therapy;
automatically identifying a second therapy program for the DBS therapy to address the acute reduction in therapy;
delivering the DBS therapy according to the second therapy program;
prompting the user to report whether the user likes or dislikes the DBS therapy delivered according to the second therapy program; and
controlling the DBS therapy based on the report by
choosing a third therapy program when the user does not like the second therapy program,
reverting to the first therapy program when the user does not like the second therapy program, or
continuing to deliver the DBS therapy according to the second therapy program when the user likes the second therapy program.

2. The method of claim 1, wherein the logged event is indicative of a symptom known to the user that may be experienced by the patient and that is known to potentially cause the acute reduction in therapy.

3. The method of claim 2, wherein the automatically identified second therapy program is an accessible program for targeting the symptom.

4. The method of claim 1, wherein the logged event is indicative of a side effect known to the user that may be experienced by the patient and that is known to the user to potentially cause the acute reduction in therapy.

5. The method of claim 4, wherein the automatically identified second therapy program is an accessible program for targeting the side effect.

6. The method of claim 1, wherein the logged event from the user is indicative of an activity that corresponds to a set of symptoms that cause the first therapy to be suboptimal.

7. The method of claim 6, further comprising automatically requesting additional information when the logged event is received, wherein the additional information is requested using user-answered queries or is received via user-provided free text.

8. The method of claim 1, wherein a weighted summary map combines weights of clinical effects data into a cumulative score, and the second therapy program is automatically identified by altering the weights of symptoms in the clinical effects data, and identifying a program with a best estimated score.

9. The method of claim 8, wherein the second therapy program is automatically identified by placing a side effect barrier on the cumulative map, the automatically identified second therapy program has a best estimated score remaining outside of a side effect region.

10. The method of claim 1, further comprising altering a workflow to simplify patient interaction when the logged event is repeatedly received.

11. The method of claim 10, wherein the workflow is altered by introducing a schedule for delivering the DBS according to the second therapy schedule.

12. The method of claim 1, further comprising determining if a program limit has been reached the DBS therapy is delivered according to another therapy program, and only delivering the DBS therapy according to the other therapy program when a program limit has not been reached.

13. The method of claim 1, wherein the user includes at least one of the patient or a caregiver for the patient.

14. The method of claim 1, further comprising:
receiving a report on the second therapy, by prompting the user or automatically sensing a detectable response, within a first time period after beginning to deliver the DBS therapy according to the second therapy, and continuing to deliver the DBS therapy according to the second therapy program when the report is good; and
determining whether to continue to deliver the DBS therapy according to the second therapy program within a second time period after beginning to deliver the DBS therapy based on another report by prompting the user or automatically sensing the detectable response or another detectable response, wherein the second time period is longer than the first time period.

15. The method of claim 14, wherein at least one of the first time period or the second time period depends on an event type for the logged event.

16. The method of claim 14, further comprising adding at least one prompt for the user to report on the second therapy within at least one time period longer than the second time period.

17. The method of claim 1, further comprising receiving additional logged or sensed events and using the logged or sensed events to identify patterns of therapy reduction and develop event-related adjustments to the DBS therapy for the identified patterns.

18. A non-transitory machine-readable medium including instructions, which when executed by a machine, cause the machine to perform a method comprising:
delivering a deep brain stimulation (DBS) therapy to a patient according to a first therapy program;
receiving a logged event from a user or sensor indicative of an acute reduction in therapy;
automatically identifying a second therapy program for the DBS therapy to address the acute reduction in therapy;
delivering the DBS therapy according to the second therapy program;
prompting the user to report whether the user likes or dislikes the DBS therapy delivered according to the second therapy program; and
controlling the DBS therapy based on the report by
choosing a third therapy program when the user does not like the second therapy program,
reverting to the first therapy program when the user does not like the second therapy program, or
continuing to deliver the DBS therapy according to the second therapy program when the user likes the second therapy program.

19. The non-transitory machine-readable medium of claim 18, wherein the method performed by the machine further comprises:
prompting the user to report on the second therapy within a first time period after beginning to deliver the DBS therapy according to the second therapy, and continuing to deliver the DBS therapy according to the second therapy program when the report is good; and
prompting the user to report on the second therapy within a second time period after beginning to deliver the DBS therapy according to the second therapy to determine whether to continue to deliver the DBS therapy according to the second therapy program, wherein the second time period is longer than the first time period.

20. A system, comprising:
a deep brain stimulator (DBS) configured to deliver a DBS therapy to a patient according to a first therapy program; and
a processing system configured for use to:
receive a logged event from a user or sensor indicative of an acute reduction in therapy;
automatically identify a second therapy program for the DBS therapy to address the acute reduction in therapy;
deliver the DBS therapy according to the second therapy program;
prompt the user to report whether the user likes or dislikes the DBS therapy delivered according to the second therapy program; and
control the DBS therapy based on the report by
choosing a third therapy program when the user does not like the second therapy program,
reverting to the first therapy program when the user does not like the second therapy program, or
continuing to deliver the DBS therapy according to the second therapy program when the user likes the second therapy program.
